SEQUENCE LISTING (1 sequence)

SEQ ID NO: 1 - DNA, 510 bp, Lactobacillus plantarum BB9
tggagtttgatcctggctcaggacgaacgctggcggcgtgcctaatacatgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagyttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaa

(19) United States
(12) Patent Application Publication
(10) Pub. No.: US 2011/0117629 A1
(43) Pub. Date: May 19, 2011

(54) LACTOBACILLUS PLANTARUM BB9 CAPABLE OF ADHERING TO GASTROINTESTINAL TRACT AND CHOLESTEROL REMOVAL

(75) Inventors: Zhen-Mei Lin, Caotun Township (TW); Mei-Gi Huang, Caotun Township (TW); Wen-Hsin Lin, Caotun Township (TW)

(73) Assignee: FAMILY MEDICINE INTERANTIONAL CO., LTD., Caotun Township (TW)

(21) Appl. No.: 12/622,428

(22) Filed: Nov. 19, 2009

(51) Int. Cl. C12N 1/20

(52) U.S. Cl. 435/252.9

(57) ABSTRACT

A Lactobacillus plantarum BB9 capable of adhering to gastrointestinal tract and cholesterol removal is isolated from fruits and exhibits high BSH activity. In-vitro tests demonstrate that Lactobacillus plantarum BB9 has good acid and bile tolerance, and strong ability to adhere to intestinal cells. In-vivo tests show that hamsters fed high cholesterol diets added with BB9 strain have cholesterol and triglycerides in blood and liver effectively reduced, and their HDL-c/LDL-c ratios in blood are significantly higher than those of hamsters fed Lactobacillus acidophilus ATCC 43121 strain. It is hoped that the excellent acid and bile tolerance and intestinal adherence of the lactobacillus strain provided herein could produce cholesterol-lowering effect in humans.

### BACKGROUND OF THE INVENTION

1. Field of the Invention

The present invention relates to a Lactobacillus plantarum BB9 capable of adhering to gastrointestinal tract and cholesterol removal for applying in the fields of pharmaceuticals, health food, weight loss diet, health supplement products, food products and beverages.

2. Description of Related Art

High blood lipid level is one of the major causes of cardiovascular disease and atherosclerosis. The levels of triglycerides (TG), high-density lipoprotein cholesterol (HDL-c) and low-density lipoprotein cholesterol (LDL-c) are also highly associated with cardiovascular disease. Clinically, blood cholesterol level is a good predictor of the risk of cardiovascular diseases. According to the U.S. National Cholesterol Education Program (NCEP), total blood cholesterol over 240 mg/dl is defined as high cholesterol level, between 200˜239 mg/dl is considered borderline-high risk, and below 200 mg/dl is desirable level, which means high cholesterol increases the risks of cardiovascular diseases and mortality rate.

Lactobacilli are commonly existed in the intestinal microflora of humans and animals and offer health benefits. Thus they are often used as probiotics and are added in food products for health enhancement. Mann and Spoerry observed back in 1974 that the concentration in the blood of the Maasai tribesmen of Africa has lowered after consumption of large amount of lactobacillus-fermented milk. Since then, the correlation between Lactobacillus and cholesterol has become a research topic. So far, the mechanism of how probiotic Lactobacillus lowers cholesterol has not yet been clearly discovered. Some studies show that Lactobacillus reduces cholesterol level by directly adhering to or precipitating cholesterol. Further studies show that the cell membrane of Lactobacillus spp. and Bifidobacterium spp. could bind cholesterol and then assimilate and metabolize cholesterol into substances needed by bacteria.

Although studies have shown the cholesterol-lowering activity of a variety of lactobacillus strains as described above, and a strain Lactobacillus acidophilus ATCC 43121 has been shown to possess hypocholesterolemic action, the biotechnology industry continues to delve into the subject in the hope to develop Lactobacillus strains with strong cholesterol-lowering activity given the significant role of cholesterol in cardiovascular disease. Similarly, the inventor of this invention has been endeavoring to develop a Lactobacillus strain that provides better cholesterol-lowering effect in the hope to offer the large population of cardiovascular patients or candidates for cardiovascular diseases a new lactobacillus option that is safe to ingest and helps lower or control cholesterol concentration in the body, thereby reducing the risks and clinical symptoms of cardiovascular diseases.

### SUMMARY OF THE INVENTION

The primary object of the present invention is to provide a Lactobacillus plantarum BB9 capable of adhering to gastrointestinal tract and removing cholesterol.

The Lactobacillus plantarum BB9 of the invention is isolated from fruits and is deposited at the DSMZ (Deutsche Sammlung von Mikroorganismen and Zellkulturen GmbH) in Germany under accession number DSM 22774 (part sequence of its 16SrDNA is shown in FIG. 8). In-vitro tests show that this strain has good acid and bile tolerance and strong ability to adhere to intestinal cells. In-vivo tests show that hamsters fed with high-calorie and high-cholesterol diet have their cholesterol and triglycerides in blood and liver effectively lowered if they were simultaneously fed with 1×10⁹ CFU/g Lactobacillus plantarum BB9, and the HDL-c/LDL-c ratio in their blood is also higher than that of the other groups. In the monitoring of body weight, it is found that the weight gained in hamster group fed with Lactobacillus plantarum BB9 is less than that of other groups. The experiments also found out that the cholesterol-lowering effect in hamsters fed with Lactobacillus plantarum BB9 is more pronounced than that in hamsters fed with the same dosage of Lactobacillus acidophilus ATCC 43121, a strain that is internationally recognized for its cholesterol-lowering effect. The Lactobacillus plantarum BB9 has been shown to effectively reduce the levels of cholesterol and triglycerides in blood and liver. Hence the Lactobacillus plantarum BB9 will aid considerably in the prevention or treatment of cardiovascular diseases in animals and humans.

### BRIEF DESCRIPTION OF THE DRAWINGS

FIG. 1 shows blood total cholesterol concentration of hamsters fed on high-cholesterol diet and different dose of freeze-dried Lactobacillus plantarum BB9.

FIG. 2 shows blood triglycerides concentration of hamsters fed on high-cholesterol diet and different dose of freeze-dried Lactobacillus plantarum BB9.

FIG. 3 shows blood HDL-c/LDL-c ratio of hamsters fed on high-cholesterol diet and different dose of freeze-dried Lactobacillus plantarum BB9.

FIG. 4 shows blood HDL-c and LDL-c levels of hamsters fed on high-cholesterol diet and different dose of freeze-dried Lactobacillus plantarum BB9.

FIG. 5 shows liver cholesterol concentration of hamsters fed on high-cholesterol diet and different dose of freeze-dried Lactobacillus plantarum BB9.

FIG. 6 shows liver triglycerides concentration of hamsters fed on high-cholesterol diet and different dose of freeze-dried Lactobacillus plantarum BB9.

FIG. 7 shows a pulsed-field gel electrophoretic DNA fingerprinting of Lactobacillus plantarum BB9 of the invention.

FIG. 8 is a schematic view showing the 16S rDNA sequence of Lactobacillus plantarum BB9 of the invention.

### DETAILED DESCRIPTION OF THE INVENTION

The present invention relates to a Lactobacillus plantarum BB9 capable of adhering to gastrointestinal tract and removing cholesterol. Its sampling method, experimental steps and results are described below:

Sampling method and experimental steps:

### 1. Collection of Lactobacilli

The lactobacilli used in testing came from Bioresource Collection and Research Center (BCRC, Taiwan, Hsinchu), feces of newborns and adults from hospitals and postpartum service centers in central Taiwan, fruits, traditional pickled food from the local markets, and animal sources. The isolated strains were subjected to catalase test, Gram's stain, microscopy and motility analysis and were preliminarily identified to be lactobacilli. All isolated lactobacillus strains were cultured in lactobacilli MRS broth (containing 0.02% L-cysteine) under 37° C. for 24 hours and then stored in MRS broth added glycerol (15%, v/v) at −70° C. in a freezer.

### 2. Digestive Tract Simulation Tests

(1) Acid Tolerance Test

The method of Zavaglia et al. (1998) was modified to study the acid tolerance of lactobacilli. The lactobacillus strains were cultured overnight and 1 ml of each culture suspension was washed once with pH 7.2 phosphate buffer solution (PBS) and then re-suspended with DI water. For each lactobacillus strain, 100 μl of resulting suspension was inoculated into a series of tubes containing 9.9 ml sterile PBS at various pH values (pH 2.0, 2.5 and 7.2). The thoroughly mixed lactobacillus suspension and buffer solution was placed in a 37° C., 80 rpm incubator for three hours and 1 ml of suspension was taken out immediately after to undergo serial dilution with pH 7.2 PBS. The lactobacillus suspension was cultured in MRS agar at 37° C. for 48 hours and the number of viable lactobacillus colony was counted.

(2) Bile Tolerance Test

The methods of Lin et al. (2006) and Gilliland and Walker (1990) were modified to study the effect of bile on lactobacilli growth. The lactobacillus strains were cultured overnight and 100 μl of each culture suspension was inoculated into a tube containing 10 ml MRS broth added with 0.3% (w/v) ox gall (Sigma) or without. The inoculated tubes were incubated at 37° C. for 4 hours and 1 ml of the culture suspension was inoculated into a tube containing 9 ml pH 7.2 PBS for serial dilution. The resulting suspension was cultured in MRS agar and the growth of lactobacilli in the presence of bile salt was evaluated using the pour plate count method. In addition, at the intervals of 0, 2, and 4 hours, 1 ml aliquots of suspension were removed for centrifuge and re-suspended with PBS. The absorbance of resulting suspension was then measured at 660 nm (OD660) using a spectrophotometer and bile tolerance (%) of the lactobacillus strain was calculated using the following formula:

Bile tolerance (%)=(Increment of OD 600 nm in MRS broth with bile salt/increment of OD 600 nm in MRS broth without bile salt)×100

### 3. Adhesion Assay

(1) Preparation of Lactobacillus Culture

All lactobacillus strains to be tested were washed twice with MRS broth and then inoculated in 5 ml MRS broth for culture. 1 ml of the culture suspension was centrifuged at 6000 rpm for 10 minutes after 24 hours. The suspension was then washed twice with PBS (pH 7.2) for adhesion assay.

(2) Adhesion Assay

Adhesion assay was carried out in reference to the methods of Lin et al. (2006) and Sarem et al. (1996).

a. Human intestinal Caco-2 cell line was treated with 1 ml 0.05% Trypsin for 5 minutes. The culture flask was then tapped gently to dislodge the cells. 1×10⁴ cells were added to each of the 96-well cell culture plates, and the medium in the wells was replaced with 200 μl of fresh DMEM daily.

b. Next, 20 μl of lactobacillus suspension was added to each well and cultured for 1 hour to allow lactobacilli to adhere to the cells.

c. 1 hour after the lactobacilli has adhered to the cells, the culture medium in the wells was discarded and wells were washed with PBS five times to remove non-adherent lactobacilli. Each well was added 100 μl 10% formalin to fixate the cells and bacteria for 30 minutes and then washed with PBS three times. Finally, 100 μl crystal violet was added for staining, and was quickly rinsed off with a small amount of 75% alcohol to remove the dye on cells after 5 minutes.

d. Phase-contrast microscope was used to observe the adherence of lactobacilli to intestinal epithelial cells and the number of adherent lactobacilli per 50 cells was counted in randomized microscopic fields. Finally, the average adherent lactobacilli per cell were calculated.

### 4. In-Vivo Animal Testing

In-vivo animal testing was carried out using a modified version of Usman and Hosono (2000).

(1) Test Animal

Syrian male hamsters used for the tests were purchased from the National Laboratory Animal Breeding & Research Center in Taiwan. The hamsters were 7˜8 weeks old and weighed 82-98 grams, averaging 95.3 grams before the test. Each hamster was separately bred in individual ventilated cage (IVC) under 25° C. and the light/dark cycle of 12 hours in light and 12 hours in darkness. Feeds and sterile RO water were provided ad libitum. During the one-week acclimatization period, the hamsters were on AIN-93 diet. But feeds with different proportions of ingredients were provided after the hamsters were grouped as described below.

(2) Grouping of Hamsters and Preparation of Feed Samples

The hamsters were randomly divided into 5 groups after one week of breeding. Each group has 6 hamsters as shown below. The feed formulation used in the testing was based on AIN-76 and further included the ingredients of casein, cornstarch, soybean oil, fiber, mineral mix, vitamin mixture, sucrose, cholesterol and cholic acid. Except for the blank control group, the feeds for the other groups had the proportion of soybean oil increased to 15% and 0.5% cholesterol added to induce hypercholesterolemia. The feeds for the test groups were added with freeze-dried lactobacilli in powder form with the proportion of cornstarch reduced. All feeds were stored at 4° C. Fresh feed and sterile RO water were provided daily.

A: Blank control group (Blank)—Hamsters in this group were fed with AIN-93 without the addition of cholesterol or any lactobacillus strain.

B: High-Cholesterol group (HC)—Hamsters in this group were fed with feeds having an additional 0.5% cholesterol and 10% lactobacilli-free freeze-dried protectant powder. The proportion of cornstarch in the standard AIN-93 formulation was also properly adjusted.

C: High cholesterol+Lactobacillus acidophilus ATCC 43121-1×10⁹ CFU/g (HC+ATCC 43121-9′ group)—Hamsters in this group were fed with feeds having an additional 0.5% of cholesterol and freeze-dried powder containing 1% Lactobacillus acidophilus ATCC 43121 to obtain a final concentration of 1×10⁹ CFU/g. The proportion of cornstarch in the standard AIN-93 formulation was also properly adjusted.

D: High cholesterol+Lactobacillus plantarum BB9-1×10⁹ CFU/g (HC+BB9-9′ group)—Hamsters in this group were fed with feeds having an additional 0.5% of cholesterol and freeze-dried power containing 1% Lactobacillus plantarum BB9 to obtain a final concentration of 1×10⁹ CFU/g. The proportion of cornstarch in the standard AIN-93 formulation was also properly adjusted.

E: High cholesterol+Lactobacillus plantarum BB9-1×10¹⁰ CFU/g (HC+BB9-10′ group)—Hamsters in this group were fed with feeds having an additional 0.5% of cholesterol and freeze-dried power containing 10% Lactobacillus plantarum BB9 to obtain a final concentration of 1×10¹⁰ CFU/g. The proportion of cornstarch in the standard AIN-93 formulation was also properly adjusted.

(3) Body Weight Measuring and Sampling

The body weights of hamsters were recorded every week. After four weeks of breeding, the hamsters were sacrificed after an overnight fasting. Blood was sampled from the heart immediately after sacrifice, which was mixed with a small amount of anticoagulant (0.68 g/l) and then stored at 4° C. In addition, liver was removed, washed and irrigated with saline water, and then wiped clean. Exactly 1 gram of liver was weighted and placed in a sample vial. The vial was added 5 ml of Folch solution (chloroform: methanol=2:1; v/v) and was protected from light and immediately stored at −80° C. in a freezer.

(4) Sample Preparation and Lipid Profile Analysis

The collected blood was centrifuged at 3000 rpm for 10 minutes. The supernatant was aspirated and immediately frozen at −80° C. until lipid analysis was conducted. Liver in the sample vial was crushed with a homogenizer and agitated under room temperature for 20 minutes to extract the lipids. The extractant was filtered using Whatman No. 2 filter and then added in Folch solution (chloroform: methanol=2:1; v/v) to bring the volume to 10 ml. The extractant was stored at −80° C. until analysis. Lipid analysis was conducted according to the procedures described below:

(a) Assay of Total Cholesterol

Cholesterol content in the samples was assayed using CHOD-PAP sold on the market by the following steps: Add coloring agent to 10 μL of blood sample and water bath the sample at 37° C. for 5 minutes. Use cholesterol esterase to release all cholesterols in the sample. Treat the sample with cholesterol oxidase to yield H₂O₂. By the action of perioxidase in the reagent, H₂O₂ produced colored quinonimine by reacting with 4-aminoantipyrine and salicyclic alcohol. The absorbance of quinonimine at 500 nm was measured and compared with that of the standard solution to obtain the content of cholesterol in the sample.

(b) Assay of Triglycerides

Triglyceride content in the sample was assayed using GPO-PAP sold on the market by the following steps: Add coloring agent to 10 μL of blood sample and water bath the sample at 37° C. for 15 minutes. Triglycerides in the sample were hydrolyzed to glycerol and free fatty acids by lipase. Under the action of glycerol kinase and glycerol phosphate oxidase, glycerol was oxidized to yield H₂O₂. By the action of perioxidase in the reagent, H₂O₂ produced colored quinonimine by reacting with 4-aminoantipyrine and salicyclic alcohol. The absorbance of quinonimine at 500 nm was measured and compared with that of standard solution to obtain the content of triglycerides in the sample.

(c) Assay of Blood HDL-Cholesterol

DL-c content in blood was assayed using blood lipoprotein cholesterol test kit sold on the market (Randox Laboratories) by the following steps: After adding precipitating agent (containing 1.4 mmol/l phosphotungstic acid, 8.6 mmol/1 magnesium chloride) to the sample, add coloring agent to 10 μL of blood sample and water bath the sample at 37° C. for 15 minutes. Add the reagent for measuring total cholesterol and measure the absorbance at OD 500 nm. Compare the result with that of standard solution to obtain the HDL-c content in the sample.

(d) Assay of Blood LDL-Cholesterol

LDL-c content in blood was assayed using blood lipoprotein cholesterol test kit sold on the market (Randox Laboratories) by the following steps: After adding precipitating agent (containing 0.68 g/l heparin, 64 mmol/l sodium citrate, and stabilizer) to the sample, add coloring agent to 10 μL of blood sample and water bath the sample at 37° C. for 15 minutes. Add the reagent for measuring blood cholesterol. Measure absorbance at OD 500 nm. Compare the result with that of standard solution to obtain the LDL-c content in the sample.

### 5. Strain Identification

(1) 16S rDNA Sequence Analysis

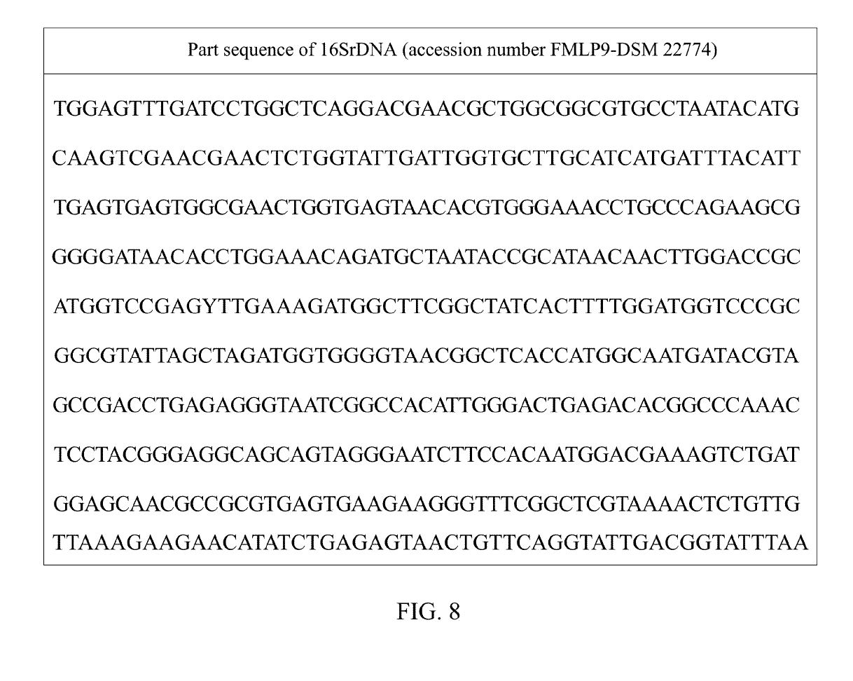

The Food Industry Research and Development Institute (FIRDI) in Hsinchu, Taiwan was commissioned to conduct 16S rDNA sequence analysis of BB9 (as shown in FIG. 8) to determine the species of BB9 deposited at DSMZ (Deutsche Sammlung von Mikroorganismen and Zellkulturen GmbH) in Germany under accession number DSM 22774. The species identification report prepared by the FIRDI is enclosed.

(2) API 50CHL Test Kit

Aside from DNA sequence analysis, the FIRDI was also commissioned to conduct API 50 CHL test to further confirm the species of BB9. This species identification report is also enclosed with the patent application.

(3) Pulsed Field Gel Electrophoresis (PFGE) Pulse field gel electrophoresis and different restriction enzymes were used to analyze the genomic DNA of isolated lactobacillus BB9 so as to identify its DNA fingerprinting.

### 6. Statistics

Each of the above tests was repeated three times. The test results were put to statistical analysis using SPSS. The results were first subjected to one-way ANOVA (analysis of variance) test to determine the presence of significant differences, and then were subjected to Duncan's multiple range test to determine the significance of differences between the groups at P<0.05.

### Test Results

### 1. Acid Tolerance Test

In the acid tolerance test, PBS was adjusted to pH 2.0, 2.5 and 3.2 using hydrochloric acid to evaluate the survival of lactobacilli in the acidic gastric environment. As shown in the table below, under our test model, BB9 strain exhibited higher survival rate and hence higher acid tolerance than the other strains at pH 2.0.

Acid Tolerance of Lactobacilli Strains
Survival rate (log CFU/ml)

| Strain | pH 7.2 | pH 2.0 | pH 2.5 | pH 3.2 |
|---|---|---|---|---|
| BB9 | 7.28 ± 0.04 | 4.27 ± 0.22 | 4.84 ± 0.13 | 7.85 ± 0.10 |
| AC9 | 7.12 ± 0.09 | <1 | 2.06 ± 0.25 | 6.22 ± 0.08 |
| V2 | 7.48 ± 0.16 | 2.90 ± 0.51 | 3.16 ± 0.22 | 7.35 ± 0.17 |
| IP3 | 7.55 ± 0.13 | 3.21 ± 0.15 | 3.87 ± 0.09 | 7.42 ± 0.05 |
| ATCC43121 | 7.17 ± 0.12 | <1 | 3.23 ± 0.26 | 6.44 ± 0.13 |
| IC5 | 7.58 ± 0.02 | 3.02 ± 0.04 | 4.08 ± 0.05 | 5.33 ± 0.02 |
| R39 | 7.69 ± 0.06 | 3.01 ± 0.05 | 4.36 ± 0.06 | 7.59 ± 0.06 |
| AE5 | 7.38 ± 0.03 | 3.18 ± 0.04 | 4.48 ± 0.07 | 5.46 ± 0.01 |
| LP33 | 7.34 ± 0.23 | <1 | 3.40 ± 0.24 | 6.57 ± 0.21 |
| AH7 | 7.45 ± 0.06 | 4.16 ± 0.04 | 4.29 ± 0.06 | 7.50 ± 0.04 |
| BB3 | 7.60 ± 0.02 | 2.73 ± 0.10 | 4.77 ± 0.08 | 7.52 ± 0.08 |
| V9 | 7.5 ± 0.04 | 3.48 ± 0.02 | 4.31 ± 0.06 | 6.57 ± 0.03 |
| AY5 | 7.27 ± 0.12 | <1 | 3.41 ± 0.29 | 6.74 ± 0.11 |
| BF6 | 7.5 ± 0.04 | 3.48 ± 0.02 | 4.31 ± 0.06 | 6.57 ± 0.03 |

### 2. Bile Tolerance Test

In bile tolerance test, 0.3% ox gall was added to MRS broth to evaluate the growth of lactobacilli in a bile environment. As shown in the table below (Colony Growth Analysis of Lactobacillus Strains in MRS Broth Containing 0.3% Bile Salt), when comparing the colony growth of tested lactobacilli in culture mediums with or without bile salt, it is apparent that 0.3% bile salt environment could inhibit the growth of lactobacilli, resulting in lower colony-forming units (CFU). Of the lactobacilli tested, BB9 exhibited the best bile tolerance with log CFU/ml reaching 8.54 after four hours in a bile salt environment. Summing up the results of acid tolerance and bile tolerance tests, only BB9 strain exhibited better tolerance to acid and bile salt, whereas other strains tested showed poor resistance to acid and the growth was inhibited considerably in a bile environment.

Colony Growth Analysis of Lactobacillus Strains in MRS Broth
Containing 0.3% Bile Salt

| | Colony growth (log CFU/ml) | | |
|---|---|---|---|
| | | 4-hour culture | |
| Strain | 0-hour culture | 0% | 0.3% |
| BB9 | 7.69 ± 0.05 | 9.43 ± 0.12 | 8.54 ± 0.14 |
| AC9 | 7.35 ± 0.13 | 8.93 ± 0.09 | 8.06 ± 0.11 |
| V2 | 6.85 ± 0.09 | 9.49 ± 0.12 | 7.64 ± 0.07 |
| IP3 | 7.62 ± 0.14 | 9.34 ± 0.06 | 7.35 ± 0.12 |
| ATCC43121 | 7.47 ± 0.10 | 8.85 ± 0.06 | 7.86 ± 0.09 |
| IC5 | 7.16 ± 0.35 | 9.22 ± 0.18 | 7.24 ± 0.19 |
| R39 | 7.41 ± 0.15 | 9.09 ± 0.03 | 7.26 ± 0.20 |
| AE5 | 6.80 ± 0.14 | 9.29 ± 0.19 | 7.14 ± 0.09 |
| LP33 | 7.45 ± 0.20 | 9.14 ± 0.11 | 7.36 ± 0.16 |
| AH7 | 7.12 ± 0.10 | 8.89 ± 0.21 | 8.23 ± 0.17 |
| BB3 | 6.90 ± 0.05 | 9.10 ± 0.14 | 8.01 ± 0.14 |
| V9 | 7.34 ± 0.17 | 8.99 ± 0.13 | 8.16 ± 0.15 |
| AY5 | 6.95 ± 0.05 | 9.12 ± 0.08 | 7.01 ± 0.03 |
| BF6 | 7.55 ± 0.13 | 9.28 ± 0.16 | 7.31 ± 0.09 |

### 3. Adhesion Assay

As shown in the table below (Adhesion Assay of Lactobacilli to Caco-2 Intestinal Cells), the BB9 strain of the present invention exhibits strong adherence to human intestinal cells Caco-2, exceeding 30 CFU/cell.

Adhesion Test of Lactobacilli to Caco-2 Intestinal Cells

| | Adherence |
|---|---|
| Strain | (CFU/cell) |
| BB9 | >30 |
| AC9 | 11.5 ± 0.3 |
| V2 | 0 |
| IP3 | 0 |
| ATCC43121 | 0 |
| IC5 | 0 |
| R39 | 7.9 ± 0.9 |
| AE5 | 0 |
| LP33 | 0 |
| AH7 | 7.3 ± 0.8 |
| BB3 | 0 |
| V9 | 0 |
| AY5 | 0 |
| BF6 | 12.8 ± 0.7 |

### 4. In-Vivo Animal Testing

During the four-week test period, no significant difference (p>0.05) was found in the average weight among the blank control group and the test groups that were fed with high cholesterol diets and the hair color of the hamsters was normal and free of hair loss. But most of the livers of sacrificed hamsters fed with high cholesterol diets showed white adipose tissue, indicating serious pathology of fatty liver.

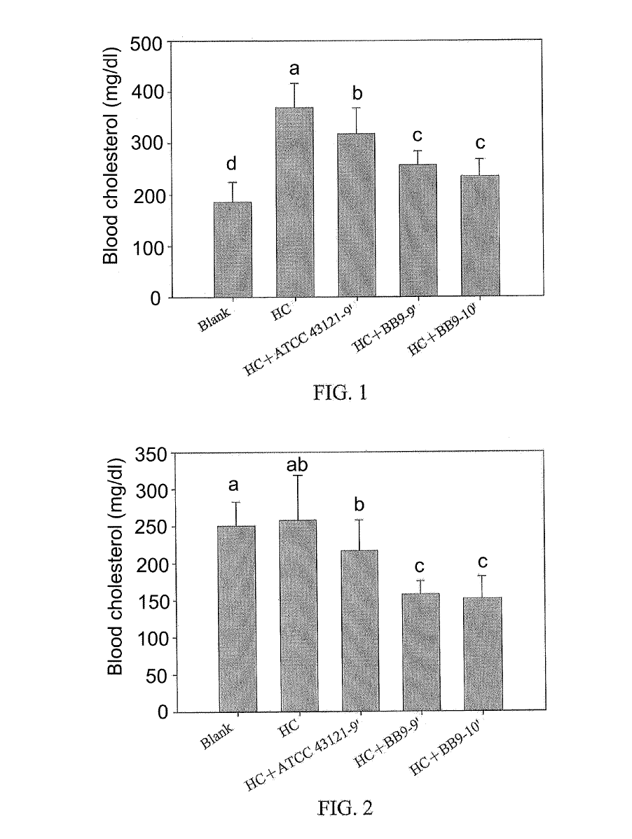

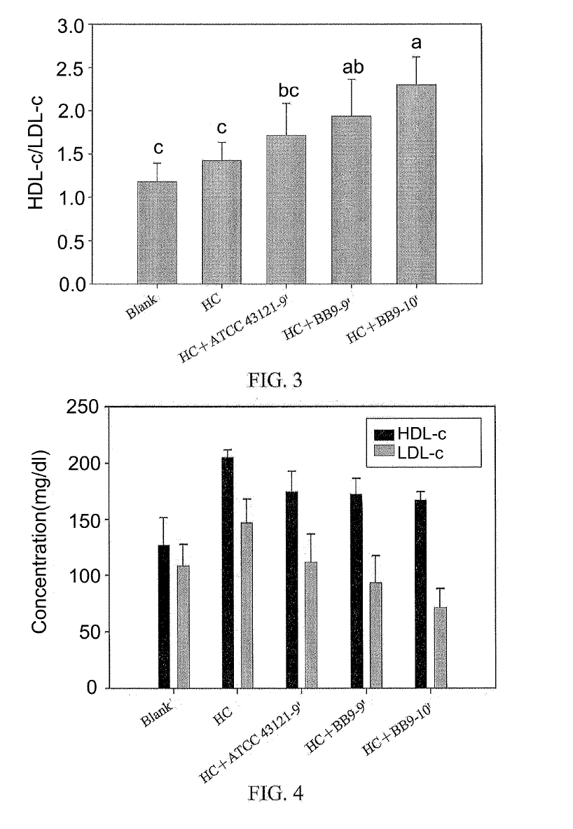

In blood lipid analysis, total cholesterol in the blood of hamster groups fed with high cholesterol diets was significantly higher (P<0.05) as compared to hamsters on normal diet (Blank group) (FIG. 1), while hamsters fed with freeze-dried Lactobacillus plantarum BB9 also showed lower cholesterol (P<0.05) with the group fed with 1×10¹⁰ CFU/g BB9 lactobacilli strain showing best cholesterol-lowering effect, which is significantly different from the result of the Lactobacillus acidophilus ATCC 43121 (1×10⁹ CFU/g) group. FIG. 2 depicts the analysis of blood triglycerides, showing that the levels of blood triglycerides in hamsters fed with 1×10⁹ or 1×10¹⁰ CFU/g Lactobacillus plantarum BB9 were significantly lower, whereas the level of blood triglycerides of the ATCC 43121 group was reduced but not significantly different from that of the blank group (P>0.05). The blood HDL-c and LDL-c levels and HDL-c/LDL-c ratios were also measured and the results are illustrated in FIG. 3 and FIG. 4. As shown, the average HDL-c/LDL-c ratios of hamster groups fed high cholesterol diets increased as compared to the blank group, but only the ratios of two hamster groups fed Lactobacillus plantarum BB9 strain showed significant difference as compared with the blank group or HC group. The average HDL-c/LDL-c ratio of the group fed 1×10¹⁰ CFU/g Lactobacillus plantarum BB9 reaching the highest at 2.3. The increase in ratio was brought about by the significant decrease in LDL-c. The ratio of Lactobacillus acidophilus ATCC 43121 group also increased as compared to the blank group, but the difference was not significant (P>0.05).

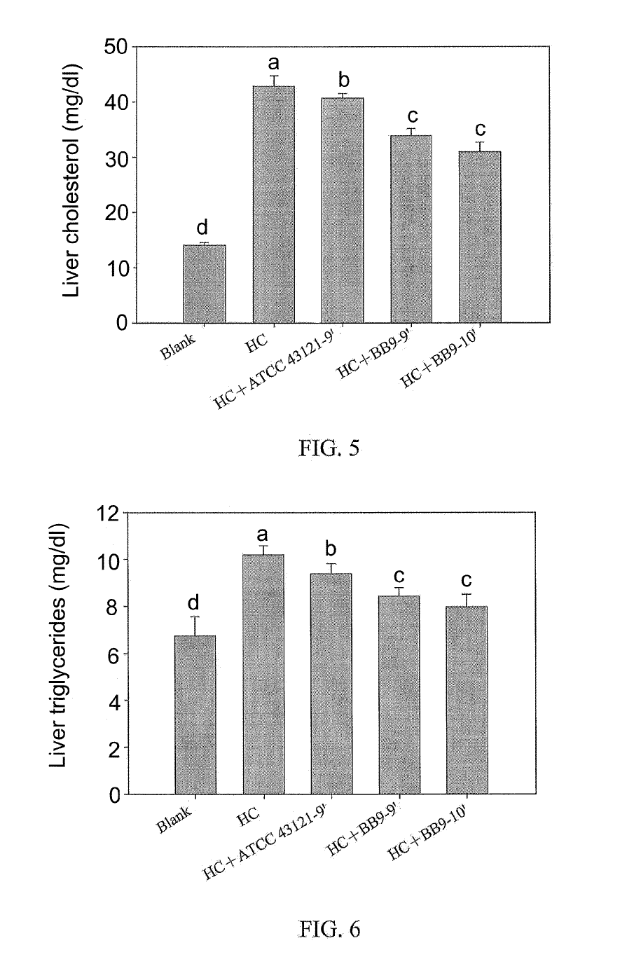

In the analysis of liver lipids, it was found that total cholesterol in the liver of hamster groups fed high cholesterol diets was significantly higher (P<0.05) as compared to hamsters on normal diet (Blank group) (FIG. 5). Hamster groups fed 1×10⁹ or 1×10¹⁰ CFU/g Lactobacillus plantarum BB9 showed significant lower cholesterol (P<0.05), whereas the ATCC 43121 group did not show much change as compared to the HC group. The results of liver triglycerides as depicted in FIG. 6 are similar to those of total cholesterol in liver showing that the ATCC 43121 group has not lowered liver triglycerides level, whereas 1×10⁹ and 1×10¹⁰ CFU/g BB9 groups showed significant decrease (P<0.05), and the lowering effect was most pronounced in the high BB9 concentration (1×10¹⁰ FU/g) group.

### 5. Strain Identification

(1) Identification of Probiotic Lactobacillus Using Restriction Enzyme Mapping

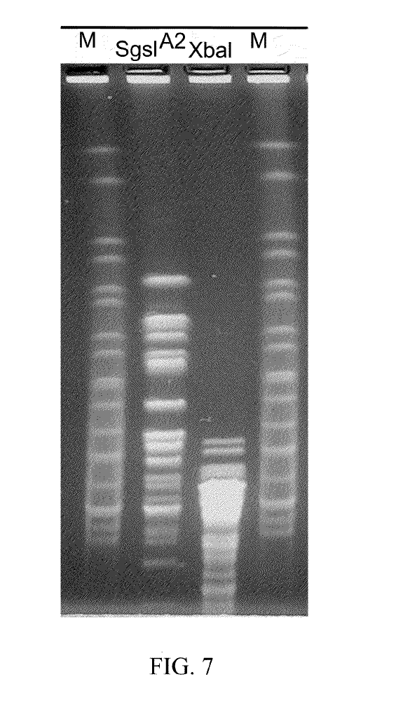

BB9 lactobacillus was treated with restriction enzymes Sgs I and Xba I, and the result is as shown in FIG. 7.

(2) 16S rDNA Sequence Analysis and API 50CHL Test

The Food Industry and Development Institute (FIRDI) in Hsinchu, Taiwan was commissioned to conduct 16S rDNA sequence analysis of BB9 (as shown in FIG. 8) and the results are presented in the enclosed species identification report. The BB9 strain is also subject to API 50 CHL test and identified as Lactobacillus plantarum.

While we have shown and described the embodiment in accordance with the present invention, it should be clear to those skilled in the art that further embodiments may be made without departing from the scope of the present invention.

What is claimed is:

1. A Lactobacillus plantarum BB9 capable of adhering to gastrointestinal tract and cholesterol removal and deposited at the DSMZ (Deutsche Sammlung von Mikroorganismen and Zellkulturen GmbH) in Germany under accession number DSM 22774.

2. The Lactobacillus plantarum BB9 capable of adhering to gastrointestinal tract and cholesterol removal as claimed in claim 1, wherein the BB9 strain is used for adhering to the cells of gastrointestinal tract of animals and humans.

3. The Lactobacillus plantarum BB9 capable of adhering to gastrointestinal tract and cholesterol removal as claimed in claim 1, wherein the BB9 is used for lowering the concentration of cholesterol in animals and humans.

4. The Lactobacillus plantarum BB9 capable of adhering to gastrointestinal tract and cholesterol removal as claimed in claim 1, wherein the BB9 is used for lowering the concentration of triglycerides in animals and humans.

5. The Lactobacillus plantarum BB9 capable of adhering to gastrointestinal tract and cholesterol removal as claimed in claim 1, wherein the BB9 is used for improving the concentration of blood lipids in animals and humans.

6. The Lactobacillus plantarum BB9 capable of adhering to gastrointestinal tract and cholesterol removal as claimed in claim 1, wherein the BB9 is applied in the fields of pharmaceuticals, health food, weight loss diet, health supplement products, food products and beverages.
